(19) United States
(12) Patent Application Publication
(10) Pub. No.: US 2005/0197385 A1
(43) Pub. Date: Sep. 8, 2005

(54) Use of rotigotine for treatment or prevention of dopaminergic neuron loss

(75) Inventors: Dieter Scheller, Neuss (DE); Frank Dressen, Vettweiss (DE)

Correspondence Address: EDWARDS & ANGELL, LLP, P.O. BOX 55874, BOSTON, MA, 02205, US

(73) Assignee: Schwarz Pharma AG

(21) Appl. No.: 11/060,997

(22) Filed: Feb. 17, 2005

Related U.S. Application Data
(63) Continuation of application No. PCT/EP04/14655, filed on Dec. 23, 2004.
(60) Provisional application No. 60/546,611, filed on Feb. 20, 2004.

(51) Int. Cl. A61K 31/381

(52) U.S. Cl. 514/438

(57) ABSTRACT

The present patent application relates to the use of Rotigotine or its salts and prodrugs for preparation of a drug for treatment or prophylaxis of dopaminergic cell loss due to diseases that are connected with an increased dopaminergic cell loss. In particular, the patent application is related to the use of Rotigotine as a drug for preventive treatment of Morbus Parkinson.

Parkinson's disease is a result of chronic progressive degeneration of neurons, the cause of which has not yet completely been clarified. Clinically, it manifests in the form of the cardinal symptoms resting tremors, rigor, bradykinesia, and postural instability.

Levodopa, dopamine agonists such as Rotigotine, Pramipexol, Bromocriptine, Ropinirol, Cabergoline, Pergolide, Apomorphine and Lisuride, Anticholinergics, NMDA antagonists, β-Blocker as well as the MAO-B inhibitor Selegeline and the COMT inhibitor Entacapone are used as medicines for relief from the motor symptoms. Most of these agents intervene in the dopamine and/or choline signal cascade and thereby symptomatically influence the Parkinson-typical movement disorders.

In the present therapy for the Parkinson's disease, treatment is initiated after the appearance of the cardinal symptoms. In general, Morbus Parkinson disease is said to be clinically evident if at least two of the four cardinal symptoms (bradykinesia, resting tremors, rigor, and postural instability) are detected and respond to L-Dopa (Hughes, J Neurol Neurosurg Psychiatry 55, 1992, 181). Unfortunately, the motor function disorders in Morbus Parkinson patients become apparent only after about 70-80% of the dopaminergic neurons in the substantia nigra (SN) are irreparably damaged (Becker et al, J Neurol 249, 2002, Suppl 3:III, 40; Hornykiewicz, Encyclopaedia of Life Science 2001, 1). Chances of a therapy with lasting effects are very bleak at that point. Hence, it is desirable to initiate the therapy as early as possible.

Current clinical observations as well as anatomical and genetic research show that diagnosis of Morbus Parkinson patients at an early stage and identification of high risk patients is possible.

Thereby, for instance, the following diagnostic markers can be used:

Biochemical markers, such as neuromelanin (Gerlach, Neurotox Res 5,2003, 35; WO 02/31499), S-100 beta (Muramatsu, Glia 42, 2003, 307), alpha synuclein (WO 03/069332; WO 00/02053) or Parkin protein (Sharma, Neurol Clin N Am 20, 2002, 759) and Semaphorin (Wo 03/007803)

Genetic markers, such as, for instance, the PARK genes 1-8 (Guttman, CMAJ 4, 2003, 168; WO 03/76658); CYP2D6-B (WO 03/012137), chromosome 2q 36-37 (Pankratz, Am J Hum Gen 72, 2003, e-pub), a-synuclein (Polymeropoulos, Science. 276, 1997, 2045) or mutations in CYP2D6-B and GSTM1 deletion (WO 03/012137).

Imaging methods, such as ultrasound examination of the SN size, possibly in combination with other procedures (Becker et al, J Neurol 249, 2002, Suppl 3:III, 40) or MRI (Hutchinson M, Raff U., J Neurol Neurosurg Psychiatry. 1999 December; 67(6):815-8).

Imaging methods such as PET or SPECT (Prunier C. Bezard E, et al., Neuroimage. 2003 July; 19(3):810-6).

Sensory disorder or behavioral abnormalities, such as disorders of sleep and smell, in particular, sleep disorder of the kind ‘REM Sleep Behavior Disorder’, (Henderson, J Neurol Neurosurg Psychiatry 74, 2003, 956) or cognitive abnormalities (Rammsayer, Int J Neurosci. 91, 1997,45).

Organic problems such as constipation (Krygowska-Wajs, Funct Neurol 15,2000,41).

Depression (Camicioli R. Drugs Today (Barc). 2002 October; 38(10):677-86).

Short duration movement anomalies, such as chorea or orthostatic abnormalities

Combinations of the aforementioned markers (Stern, Annals of Neurol 56, 2004, 169)

With that an opportunity arises for influencing the disease process at a point of time when more neurons are still there, rather than at the time of appearance of several cardinal motor symptoms of the Parkinson's disease, and thereby for protecting a quantitatively greater number of neurons. One can expect that the administration of an effective neuroprotector at an early stage will significantly delay the process of the development of the disease: The sooner the therapy is initiated, the higher are the chances of a long lasting prevention of the onset of symptoms, which degrade the quality of life.

Hence, such remedies are needed that not only influence the dopaminergic transmission and alleviate the symptoms of the Parkinson's disease in advanced stages, but also reverse, prevent, or at least significantly delay the dopaminergic neuron extinction in the early, to a great extent motor-asymptomatic, Parkinson stages (Dawson, Nature Neuroscience Supplement 5, 2002, 1058).

Rotigotine [(−)-5,6,7,8-Tetrahydro-6-[propyl[2-(2-thienyl)ethyl]amino]-1-naphthol] is known from the art as a dopamine agonist and a purely symptomatic Morbus Parkinson therapeutic agent. The WO 02/089777, for example, describes the transdermal administration of Rotigotine to Parkinson patients and the consequent improvement of the UPDRS (Unified Parkinson's Disease Rating Scale) score. The UPDRS score is an important instrument for diagnosing and monitoring the progress and/or the therapy of Morbus Parkinson patients (Fahn S, Elton R L, Members of the UPDRS Development Committee (1987) Unified Parkinson's Disease Rating Scale. In: Fahn, S, C D Marsden, D B Calne, M Goldstein (eds) Recent Developments in Parkinson's Disease. Vol. II. Macmillan Healthcare Information, Florham Park (N.J.), pp 153-163, 293-304). However, the UPDRS score registers merely the effect of an active agent on the Parkinson symptoms. It does not enable to make a statement whether the active agent influences the dopaminergic cell loss, which is the underlying cause of the symptoms.

Metman et al (Clin Neuropharmacol 24,2001, 163) describe the effect of Rotigotine on Parkinson-associated motor function disorders. The treated patients had already prominent dyskinesias, which were improved through administration of Rotigotine.

Thus, according to the current status of the technology, Rotigotine is known as a dopamine agonist for symptomatic treatment of Morbus Parkinson. However, medicines that only influence the Parkinson symptoms do not promise any advantage in the preventive treatment of Morbus Parkinson, because they have no influence on the dopaminergic cell loss and the progress and/or development of the disease.

Now, surprisingly, the experimental observations have shown that Rotigotine, which was hitherto used only for the symptomatic treatment of Parkinson's disease, has also neuroprotective characteristics or promotes the regenerative capacity of neuronal elements, so that Rotigotine can be used as a drug or prophylactic agent for prevention of dopaminergic cell loss, particularly in the very early stages of the Parkinson's disease or in case of high risk candidates.

### FIGURES

FIG. 1 shows representative examples demonstrating the neuroprotective effect of Rotigotine measured from the density of the dopamine transporters as an indication for the density of the remaining nerve ends in the striatum.

Groups 1-7 were treated in the following manner: Group 1: Untreated control; Group 2: Control group treated with the excipient solution for Rotigotine and MPTP; Group 3: MPTP treatment; Group 4: MPTP treatment plus Rotigotine 0.3 mg/kg; Group 5: MPTP treatment plus Rotigotine 1.0 mg/kg; Group 6: MPTP treatment plus Rotigotine 3.0 mg/kg; Group 7: Treatment only with Rotigotine (3.0 mg/kg)

FIG. 2 shows the dopamine transporter (DAT) binding in the dorsal and ventral part of the striatum in different groups, derived from the quantification of the DAT density according to an experiment as shown in FIG. 1. The bar graphs 1-7 correspond to the Groups 1-7 shown in FIG. 1. The groups marked with * indicate a significant decline in the DAT binding as compared to the control Group 2. The groups marked with # showed a significant maintenance of the DAT binding as compared to the MPTP-treated Group 3.

### DESCRIPTION OF THE INVENTION

Apoptotic processes are said to play an important role in the loss of dopaminergic neurons in the Parkinson pathogenesis (Barzilai, Cell Mol Neurobiol 21, 2001, 215). Hence, neuroprotective substances are desired, which can stop or even reverse the dopaminergic cell loss. Thereby, the MPTP model is considered to be predictive for the required neuroprotective characteristics (Dawson, Nature Neuroscience Supplement 5, 2002, 1058).

Surprisingly, Rotigotine shows the desired pharmacological profile in an acute as well as in subacute MPTP model. The results of the study suggest that apoptotic processes are prevented by Rotigotine.

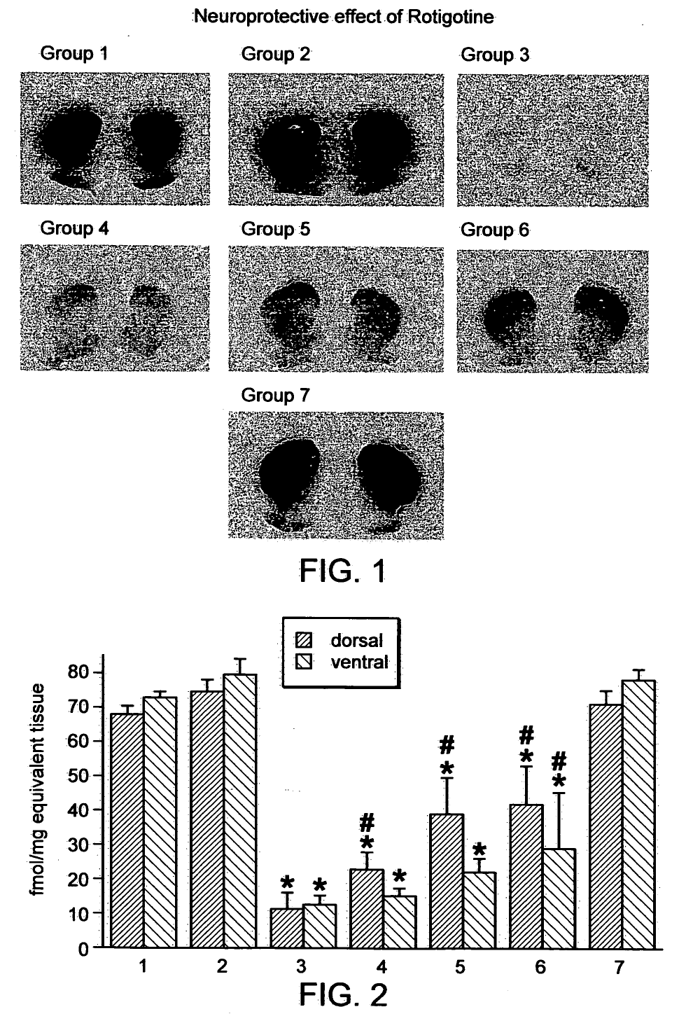

For example, Rotigotine shows neuroprotective effect in a Parkinson model of the mouse: After an acute administration of MPTP, which causes Parkinson syndrome in humans as well as in monkeys, the number of the degenerating neurons in the acute phase was measured (Table 1), and also the functional integrity of the striatum in the subacute phase was recorded by determining the density of the dopamine transporter in the terminal nerve endings (FIGS. 1 and 2). In both cases, it could be shown that Rotigotine was effective as a neuroprotective agent: On one hand, the number of degenerating neurons in the mesencephalon was reduced after the administration of Rotigotine and, on the other hand, the dopaminergic innervation of the striatum was almost completely preserved or restored.

TABLE 1
Number of degenerating neurons in the mouse,
shown with FluoroJade stain

| | Number of | |
|---|---|---|
| | degenerating | |
| Group | neurons | Stndrddev |
| 1: Excipient-treated control | 2.0 | 2.4 |
| 2: MPTP intoxication | 73.5 | 34.0 |
| 3: MPTP intoxication + Rotigotine 0.3 mg/kg | 66.7 | 30.5 |
| 4: MPTP intoxication + Rotigotine 1.0 mg/kg | 76.8 | 41.6 |
| 5: MPTP intoxication + Rotigotine 3.0 mg/kg | 34.9 | 31.9 |
| 5: MPTP excipient + Rotigotine 3.0 mg/kg | 3.8 | 4.3 |

In a pilot study, the neuroprotective effect of Rotigotine was also investigated on monkeys.

In the model used, which reflects the progressive course of the Parkinson's disease on primates, subliminal toxic doses of MPTP were injected in (macaque) monkeys for several days. The Parkinson symptoms developed in the model in a period of about 2 weeks. As soon as a certain degree of damage was reached, Rotigotine was injected daily in a formulation that built up a continuous plasma level for 24 h. The MPTP injections were stopped as soon as the motor activity was reduced by a certain degree (approx. 5 days later). The behavior of the animals was assessed on a daily basis. Six weeks after the beginning of the MPTP injection, the Rotigotine injections were stopped, and the animals were observed for another two weeks without treatment. It was observed that the motor activity of the animals had clearly improved during the treatment and also in the following washout phase.

At the end of the Rotigotine injection or washout phase, a group each of animals was killed and the condition of the basal ganglion was examined histologically and biochemically. The density of the nerve endings in the striatum had significantly increased compared to that of the untreated animals. The content of pre-pro-enkephalin, which is an indicator for the intactness of the networking in the ‘indirect pathway’ of the basal ganglion, showed a tendency toward normalization after the treatment and the washout phase.

The results show that the neuroprotective potential of Rotigotine can also be demonstrated in a primate model with the Parkinson's disease. Thus, a neuroprotective effect can also be expected in humans.

Thus, with Rotigotine, an active substance is made available for the therapy which is ideally suitable for preparation of a drug or a prophylactic agent for prevention of dopaminergic neuron loss in neurodegenerative diseases.

An object of the present application is therefore the use of Rotigotine for preparation of a drug for treatment or prevention of dopaminergic neuron loss in patients suffering from a neurodegenerative disease that is connected with an increased dopaminergic cell loss or that has a high risk of increased dopaminergic cell loss.

Increased dopaminergic neuron loss occurs regularly in Morbus Parkinson patients, however, it is also frequently observed in other neurodegenerative diseases, for example, in alpha-synucleopathies or in the Huntington's disease as well as in REM insomnias and in disorders of the sense of smell.

Compared to the use of Rotigotine hitherto, which was limited to the purely symptomatic treatment of Morbus Parkinson patients with motor function disorders, a new area of its application has now been discovered, in particular the prophylactic treatment of individuals who are at an early stage of the Parkinson's disease, or of individuals predisposed to develop Morbus Parkinson due to genetic or other risk factors. As already described further above, such individuals, compared to patients who already have the motor symptoms, profit particularly from the neuroprotective effect of Rotigotine, since the dopaminergic cell loss is stopped or retarded through administration of Rotigotine at a time, when a higher number of dopaminergic neurons are still present.

An object of the invention is therefore the use of Rotigotine or its salts and prodrugs as a medicine for the preventive treatment of the dopaminergic cell loss in individuals in whom, before the initiation of the preventive treatment, at least three of the four cardinal symptoms of Morbus Parkinson from the group bradykinesia, rigor, resting tremors, and postural instability are either not yet present at all or are only rudimentary or partial.

Thereby, the individuals can also be apparently healthy individuals, whose genetic or epidemic predisposition may not indicate a higher risk for developing Parkinson's disease.

However, treatment with Rotigotine comes in question especially in case of individuals with high risk for developing Morbus Parkinson or in case of patients in whom early clinical, clinical/chemical or clinical/physical Morbus Parkinson symptoms are evident; but these patients must not show yet two or more of the cardinal symptoms of the Parkinson's disease.

Finally Rotigotine can also be used as a neuroprotective agent, if the diagnosis is not clear, but the development of the symptoms towards Parkinson-like neurodegeneration is expected.

The prevention of the neuronal dopaminergic cell loss is needed in particular by
(a) individuals with an increased risk for Morbus Parkinson or
(b) individuals with early symptoms of Morbus Parkinson

The terms “Morbus Parkinson” and “Parkinson's disease” are used in this patent application as synonyms and include idiopathic and genetic Parkinson. The so-called Parkinson-Plus syndrome as well as the secondary Parkinsonism are to be differentiated from these.

“Cardinal symptoms” of Morbus Parkinson refer in this patent application to one or more symptoms of bradykinesia, rigor, resting tremors, and postural instability.

“Individuals with an increased risk for Morbus Parkinson” refer in this patent application particularly to individuals who still have no evident symptoms of the Parkinson's disease, but who are subject to certain risk factors.

Such risk factors can be genetic mutations (Nussbaum N E J M 348,2003, 25). For example, the PARK gene in chromosomes 6q25.2-27 (PARK2) is associated with juvenile Parkinsonism and occurs more frequently in families with autosomal recessive Parkinson inheritance (Matsumine, Am. J. Hum. Genet., 60, 1997, 588; Kitada, Nature 392, 1998, 605; Abbas, Hum. Mol. Genet. 8, 1999, 567; Tassin, Am. J. Hum. Genet., 63, 1998, 88 und Lucking, N. Engl. J. Med. 342, 2000, 1560-7). Other gene loci, for example, PARK6 and PARK7 were also found, to a certain extent, in families with juvenile, recessive inheritance of Parkinson's disease (Valente, Am. J. Hum. Genet. 68, 2001,895; van Dujin, Am. J. Hum. Genet. 69, 2001, 629). However, the PARK8 gene is associated with the late occurrence of Morbus Parkinson (WO 03/076658). Mutations in the alpha-synuclein gene (PARK1) were detected in families with juvenile, autosomal dominant transmitted Morbus Parkinson (Polymeropoulos, Science 276,1997, 2045).

Apart from the genetic predisposition, environmental influences, such as the heavy exposure, for example, to insecticides (Vanacore, Neurol Sci., September; 23 Suppl 2, 2002, S119), can also represent risk factors.

In this patent application “individuals with early Parkinson symptoms” refers particularly to those individuals in whose cases at least three of the four cardinal symptoms (rigor, resting tremors, bradykinesia, and postural instability) are not yet present or are only rudimentary or partial, but who manifest diagnostically assessable clinical, clinical/biochemical, or clinical/physical early symptoms.

Clinical/biochemical markers can be the changes in the alpha-synuclein or neuromelanin pattern (WO 02/31499). Such changes can, for example, be based on the expression of genetic variants, for instance, of alpha synuclein, the formation of aggregates or filaments, for instance, of alpha-synuclein or increased release from cellular storages, for example, from the cytoplasm of degenerating membrane cells, as with neuromelanin.

Clinical/physical early symptoms can be the structural or functional changes in the brain, which can be detected, for example, physically through PET and SPECT studies or through transcranial sonography (Becker, J Neurol 249, Suppl 3, 2002, III/40; Prunier C S, et al., neuro-image 2003 July; 19(3):810-6).

Clinical early symptoms can be smell disorders, depression, constipation, disorders of eyesight, and cognitive functions or certain forms of insomnia, particularly of the type of “REM Sleep Behavior Disorders”, but for an early detection, a combination of different tests can also be taken into consideration (Becker, J Neurol 249, Suppl 3, 2002, III/40; star, Annals of Neurol 56, 2004, 169).

As already discussed further above, 70-80% of the dopaminergic neurons of the substantia nigra have already perished at the time of the first appearance of at least two of the four cardinal symptoms. In order to effectively use the remarkable neuroprotective potential of Rotigotine, the prophylactic treatment of the patients therefore sets in favorably at a stage, when the patients have a lesser loss of dopaminergic cells of substantia nigra (SN). Therefore, such individuals are preferably treated with Rotigotine who exhibit only one or not even one of the cardinal symptoms of the Parkinson's disease in a clearly pronounced form.

Preferably such individuals are treated who have a dopaminergic cell loss in the SN of less than 70%, 60%, 50% and, particular preferably, less than 40%, 30%, 20%, or 10%.

Two scores can be used as the measure for the diagnosis and for the therapy control of the patients with already noticeable motor function disorder: The UPDRS score and the Hoehn and Yahr score.

In a preferred aspect of the invention, the prophylactic group of patients treated with Rotigotine has a modified Hoehn and Yahr score of 0 to 2, particularly preferably of 0 to 1 and very particularly preferably of 0.

TABLE 2
Modified stage determination according to Hoehn, The natural history of
Parkinson's disease in the pre-levodopa and post-levodopa eras.
Neurologic Clinics 10, 1992, 331

Stage 0 = No symptoms of disease.
Stage 1 = One-sided disease.
Stage 1.5 = One-sided and axial participation
Stage 2 = Two-sided disease without balance disorder.
Stage 2.5 = Slight two-sided disease with compensation in pull test.
Stage 3 = Slight to moderate two-sided disease.
slight postural instability; physically independent.
Stage 4 = Severe impairment, can still walk or stand without help.
Stage 5 = Without help confined to the wheelchair or bedridden.

Usually, patients with the UPDRS score, Part III (see Working Example 5) of at least 10 are classified as those who come in question for the dopaminergic therapy. However, the group of patients suitable for using the neuroprotective effect of Rotigotine has preferably a very low or not detectable motor UPDRS score (Part III). Therefore, in the sense of the present invention, the preventive treatment with Rotigotine should take place preferably in case of patients who have an UPDRS motor score of less than 10, 9, 8, 7, 6, 5, 4, 3, 2, or 1. Particularly preferably, the patients still don't have any motor disorders.

The terms “prevention”, “prophylaxis” and “preventive treatment” are used in this patent application as synonyms. They cover the administration of a drug in individuals in whom the dopaminergic cell loss is to be expected or has already occurred. These are patients in whom, in particular, at least three of the four cardinal symptoms of Morbus Parkinson (rigor, resting tremors, bradykinesia, postural instability) are not yet present or are only rudimentary or partial, so as to prevent or delay the occurrence or significant occurrence of the motor function symptoms of the Parkinson's disease, or of further dopaminergic neuron loss, particularly in the substantia nigra. Preferably, the individuals to be given prophylactic treatment should not show yet any of the cardinal symptoms in a distinctly pronounced form. The individuals to be given the prophylactic treatment have preferably an UPDRS score of less than 10, less than 9, 8, 7 or 6 and, particularly preferably, less than 5, 4, 3, 2, or 1.

In this patent application “prodrugs” of Rotigotine refer in particular to compounds that are split, transformed, or metabolized in the human body, particularly in the plasma, or during the passage through the skin or mucous membrane into a therapeutically effective quantity of Rotigotine.

Rotigotine has the following formula

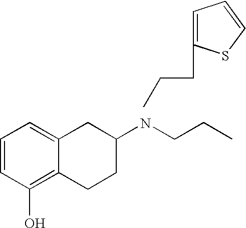

As a prodrug, derivatives of the phenolic hydroxy group are therefore come especially in question, for example, esters, such as aryl-carbonyl ester, alkyl-carbonyl ester, or cycloalkyl-carbonyl ester, particularly alkyl-carbonyl ester and cycloalkyl-carbonyl ester with up to 6 carbon atoms; carbonates; carbamates; acetals; ketals; acyloxy alkyl ether; phosphates; phosphonates; sulfates; sulfonates; thiocarbonyl ester; oxythiocarbonyl ester; thiocarbamates; ethers and silyl ethers.

The term “alkyl-carbonyl ester” includes compounds in which the oxygen atom of the Rotigotine is bonded with the group —C(O)-alkyl.

The term “cycloalkyl-carbonyl ester” includes compounds in which the oxygen atom of the Rotigotine is bonded with the group —C(O)-cycloalkyl.

The term “aryl-carbonyl ester” includes compounds in which the oxygen atom of the Rotigotine is bonded with the group —C(O)-aryl.

The term “carbonate” includes compounds in which the oxygen atom of the Rotigotine is bonded with the group —C(O)—O—R.

The term “carbamate” includes compounds in which the oxygen atom of the Rotigotine is bonded with the group —C(O)—NRR1, —C(O)—NH—R1, or —C(O)—NH₂.

The term “acetal” includes compounds in which the oxygen atom of the Rotigotine is bonded with the group —CH(OR)R1.

The term “ketal” includes compounds in which the oxygen atom of the Rotigotine is bonded with the group —C(OR)R1R2.

The term “acyloxyalkyl ether” includes compounds in which the oxygen atom of the Rotigotine is bonded with the group —CHR—O—C(O)—R1 or CH₂—O—C(O)—R1.

The term “phosphate” includes compounds in which the oxygen atom of the Rotigotine is bonded with the group —P(O₂H)OR.

The term “phosphonate” includes compounds in which the oxygen atom of the Rotigotine is bonded with the group —P(O₂H)R.

The term “sulfate” includes compounds in which the oxygen atom of the Rotigotine is bonded with the group —S(O)₂OR.

The term “sulfonate” includes compounds in which the oxygen atom of the Rotigotine is bonded with the group —S(O)₂R.

The term “thiocarbonyl ester” includes compounds in which the oxygen atom of the Rotigotine is bonded with the group —C(═S)—R.

The term “oxythiocarbonyl ester” includes compounds in which the oxygen atom of the Rotigotine is bonded with the group —C(═S)—O—R.

The term “thiocarbamate” includes compounds in which the oxygen atom of the Rotigotine is bonded with the group —C(═S)—N—RR1, —C(═S)—NH—R1, or —C(═S)—NH₂.

The term “ether” includes compounds in which the oxygen atom of the Rotigotine is bonded with the group —R.

In the above definitions of the prodrugs, R, R1 and R2 are each independently selected from hydrogen, alkyl, cycloalkyl, or aryl, and preferably from the group C1-6 alkyl, C3-10 cycloalkyl, and phenyl.

Alkyl” can be a branched or an unbranched alkyl group, which has preferably 1 to 10 C atoms, particularly preferably 1 to 6 C atoms. Alkyl groups can also be substituted with one or several substituents, for example, with halogen.

“Cycloalkyl” is an alkyl group that can consist only of pure ring-forming C atoms, or can optionally have other branching C atoms. Preferred chain lengths are 3-10, particularly preferably, 4-8 or 4-6 C atoms.

“Aryl” is preferably phenyl. Phenyl can possibly also be substituted at one or more positions, for example, with alcoxy, alkyl, halogen, or nitro.

The manufacture of prodrugs through reaction of Rotigotine with the corresponding reactive initial stages such as acid chlorides, acid anhydride, carbamyl chlorides, sulfonyl chlorides etc. is known to the specialists in the field of the clinical chemistry and can be referred from the related technical literature. Examples of literature references are Bundgaard: Design of prodrugs, Elsevier, Amsterdam, 1985; Higuchi and Stella: Pro-drugs as novel drug delivery systems in American Chemical Society, Washington DC, 1975; Sloan: Prodrugs—Topical and Ocular Drug Delivery, Ed: M. Dekker, 1992; Roche: Design of biopharmaceutical properties through prodrugs and analogs, Washington, DC, 1977.

Various prodrugs of the racemates of Rotigotine (N-0437) are described, for example, in Den Haas et al , Naunyn-Schmiedeberg's Arch Pharmacol 342, 1990, 655; Den Haas et al, Naunyn Schmiedeberg's Arch Pharmacol. 341, 1990, 186 and Den Haas et al, J Pharm Pharmacol 43, 1991,11.

The suitability in principle of a Rotigotine derivative as a prodrug can be determined by incubating the respective compounds under defined conditions with an enzyme cocktail, a cell homogenisate, or an enzyme containing cell fraction, and the emerging Rotigotine is measured. A suitable enzyme mixture is contained, for example, in the S 9 liver preparation of the company Gentest, Woburn, Mass., USA.

Alternatively, incubation can take place with fresh blood or plasma or even with a homogenate of the subcutaneous tissues to demonstrate the liver-independent metabolization of the prodrugs into the active component. For transdermal application, an in vitro examination of the permeation in excisioned skin is necessary. The final proof of the suitability and potential effectiveness in the disease models is obtained through determination of the 2-N-propylamino-5-hydroxytetralins, formed from the prodrug, in the plasma.

In vivo, a prodrug should set free so much Rotigotine that a therapeutic/prophylactic effective steady state concentration of Rotigotine in the plasma is achieved. Thereby, Rotigotine concentrations between 0.01 and 50 ng/mL, preferably between 0.05 ng and 20 ng/mL, and particularly preferably between 0.1 and 10 ng/mL plasma are considered effective concentrations.

Rotigotine is the S (−) enantiomer of 5,6,7,8-tetrahydro-6-[propyl[2-(2-thienyl)ethyl] amino]-1-naphthol. This means, according to the invention, that the proportion of the (R)-enantiomer, which is inactive in Parkinson models, is low in the drug. The (R)-enantiomer is present in the drug preferably with the proportion of <10 mol %, particularly preferably with the proportion of <2 mol %, and particularly preferably with a molar proportion of <1%, with respect to the total quantity of Rotigotine.

Rotigotine and its prodrugs can be present in the drug as free bases or in the form of physiologically acceptable salts, for example, in the form of the hydrochlorides.

“Physiologically acceptable salts” include non-toxic additive salts of the Rotigotine of organic or inorganic acids. A preferred example of a suitable salt is the HCl salt.

For the administration of Rotigotine and its prodrugs, different methods of application are available, from which the specialist can select and adjust the necessary dosage and the desired application interval according to the requirement, condition, and age of the patient.

A preferred type of the dispensation of Rotigotine is the transdermal administration. The form of administration can in principle be selected to be, for example, ointment, paste, spray, foil, plaster, or an iontophoretic agent.

Thereby, preferably, Rotigotine is administered in the plaster form on the skin of the patient, in which the active substance is available preferably in a matrix made from adhesive polymer, for example, from a self-adhesive polysiloxane. Examples for suitable transdermal formulations are given in WO 99/49852, WO 02/89777, WO 02/89778, WO 2004/058247 and WO 2004/012721 as well as in Working Example 1. Such a presentation form enables the adjustment of a largely constant plasma level and with it a constant dopaminergic stimulation across the entire application interval (WO 02/89778; Metman, Clinical Neuropharmacol. 24, 2001, 163).

If, on the contrary, a drug in the form of a subcutaneous or intramuscular depot form is desired, Rotigotine can be prepared and injected as a suspension, for example, as a salt crystal, for instance, as a crystalline hydrochloride, in a hydrophobic, anhydrous medium, as described in WO 02/15903 and Working Example 2.

Rotigotine can be administered in principle also in the form of microcapsules, microparticles, or implants on biodegradable polymer base, as described, for example, in WO 02/38646.

Other possible forms of the administration of Rotigotine and its prodrugs are transmucosal formulations, for example, sublingual sprays, rectal formulations, or aerosols for pulmonal administration.

Suitable dosages of Rotigotine are between 0.05 and approximately 50 mg/day, whereby, preferably, daily doses between 0.1 and 40 mgs and particularly between 0.2 and 20 mg/day are administered. The dosage can be given gradually, that is, the treatment can possibly begin with low dosages that be increased afterwards up to the maintenance dose.

To the specialist, it is clear that the dosage interval can vary depending on the applied quantity, the application type, and the daily requirement of the patient. So a transdermal application form can be conceived, for example, for once daily, three-day, or seven-day dosage, whereas a one-week, two-week, or four-week rhythm, for instance, is possible in subcutaneous or intramuscular depot injections.

In the neuroprotective form of the drug, besides Rotigotine, other active substances can be present that prevent the progress of the dopaminergic cell loss.

Examples for those substances are agents with antiapoptotic effect (Minocycline, FK-506, Cyclosporin A, zVAD) as well as neurotrophins, such as the Glial-cell-derived neurotrophic factor (GDNF).

In a combination preparation, a sequential administration can be achieved, for instance, in that the form of administration, for example an oral tablet, can have two different layers with differing release profiles for the different pharmaceutically active ingredients. To the specialist, it is clear that in context of the present invention, different forms of administration and application schemes are thinkable, which are all object of the invention.

Another object of the patent application is a kit for early diagnosis and treatment of Morbus Parkinson. Such a kit contains (a) a diagnostic agent that enables the diagnosis of Morbus Parkinson or the predisposition to develop Morbus Parkinson disease at an early or in asymptomatic stage and (b) a pharmaceutical formulation containing Rotigotine, its salts or prodrugs for treatment or prophylaxis of dopaminergic cell loss.

Such a kit can, for instance, consist of:

(a) an agent or a diagnosis kit for detection of neuromelanin

(b) a pharmaceutical formulation containing Rotigotine, its salts and prodrugs

In an another form of administration according to the invention, the kit can contain:

(a) an agent or a diagnosis kit for detection of Semaphorin 3

(b) a pharmaceutical formulation containing Rotigotine, its salts and prodrugs

In an another form of administration according to the invention, the kit can contain:

(a) an agent or a diagnosis kit for detection of alpha-synuclein and/or its aggregates

(c) a pharmaceutical formulation containing Rotigotine, its salts and prodrugs

In an another form of administration according to the invention, the kit can contain:

(a) an agent or a diagnosis kit for detection of genetic evidence of a mutation connected with the occurrence of Morbus Parkinson and/or of an allele associated with frequent occurrence of Morbus Parkinson, in particular, from the group of the PARK genes 1, 2, 3, 4, 5, 6, 7, or 8 as well as of gene loci CYP2D6-B and GSTM 1.

(b) a pharmaceutical formulation containing Rotigotine, its salts and prodrugs.

### WORKING EXAMPLES

### Working Example 1

### Rotigotine Plaster

1.8 g Rotigotine (free base) is dissolved in 2.4 g of ethanol and added to 0.4 g Kollidon 90F (dissolved in 1 g ethanol). This mixture is added to a 74% solution of silicon polymers (8.9 g BioPSA 7-4201+8.9 g BIO-PSA 7-4301 [Dow Corning]) in heptane. After addition of 2.65 g petrolether the mixture is stirred for 1 hour at 700 rpm to obtain a homogeneous dispersion. After lamination on polyester, it was dried at 50° C. The plaster weight was finally 50 g/cm2.

### Working Example 2

### Rotigotine Depot Suspension

(a) 1411.2 g Miglyol 812 was weighed in a Duran flask. 14.4 g Imwitor 312 was added to Miglyol and after that heated under stirring to 80° C. for 30 minutes. The clear solution was cooled to room temperature and filtered.

(b) 1188 g of the solution prepared as in (a) was transferred in a laboratory glass reactor, added to 12 g Rotigotine, and homogenised with a Ultra-Turrax at 10,000 rpm for 10 minutes under nitrogen. The suspension was filled from the running Ultra Turrax (2,000 rpm) into brown glass flasks.

### Working Example 3

### Subacute MPTP Model

For intoxication, mice are administered 80 mg/kg of the neurotoxin 1-methyl-4-phenyl-1,2,3,6-tetrahydro-pyridine (MPTP) (in portions of 20 mg/kg at two-hour intervals, Group 3-6 in FIGS. 1 and 2), leading to a degeneration of the neurons of the substantia nigra of about 50-60% (maximum degeneration in Group 3 in FIGS. 1 and 2). Rotigotine is administered daily in doses of 0.3, 1, or 3 mg/kg each for 7 days in the so-called ‘Slow-Release-Formulation’ (see Working Example 2) (Group 4-6 in FIGS. 1 and 2). A group of MPTP treated animals (Group 3) is given Rotigotine excipient solution (see Working Example 2 without Rotigotine HCl) and serves as reference. As control serve the Groups 1, 2, and 7, whereby Group 1 is not given any treatment, Group 2 is treated with the excipient solutions for MPTP and Rotigotine, and Group 7 is given exclusively Rotigotine. On the 8th day, the animals are killed, their brains taken out and frozen. Frozen sections are incubated at 100 pm [¹²⁵I] PE2I ([¹²⁵I]-(E)-N(3-iodoprop-2-enyl)-2β-carboxymethyl-3β-(4′-methylphenyl)-nortropane) in phosphate buffer, pH 7.4, in order to mark the quantity of the still present dopamin transporter in striatum and thereby to indicate the quantity of functioning nerve ends. Rotigotine improves the survival of the neurons and their nerve ends in dependence of the dosage. This is a clear indication of the neuroprotective properties of the substance (FIGS. 1 and 2).

### Working Example 4

### Acute MPTP Model (Including Apoptosis)

For intoxiation, mice are administered 80 mg/kg of the neurotoxin 1-methyl-4-phenyl-1,2,3,6-tetrahydro-pyridine (MPTP) (in portions of 20 mg/kg in two-hour intervals), leading to a degeneration of the neurons of the substantia nigra of about 50-60%. About 16 hours before, Rotigotine is administered in doses of 0.3, 1 or 3 mg/kg each as the so-called ‘Slow-Release-Formulation’. Diffusion and absorption latencies lead to it that Rotigotine is then optimally available if MPTP is administered. After 24 hours the animals are killed and the brains fixed. The brain sections are stained with FluoroJade for identification of degenerated cells. The immunohistochemical marking of tyrosine-hydroxylase served the purpose of identification of dopaminergic neurons. The staining of tyrosine hydroxylase does not show any difference between the treated and untreated animals; the staining with FluoroJade shows a large number of degenerated neurons; the neurons are, however, not yet completely perished; this suggests that the cell loss is apoptotic. The number of the degenerated neurons is about 50% less after the application of Rotigotine; this further demonstrates the neuroprotective property of the substance (Table 1).

### Working Example 5

### Determination of the Motor Function UPDRS Scores

The motor function UPDRS score (Part III of UPDRS Scores) is determined from the results of the examination of the patients by means of the criteria 18-31 reproduced in the following Table 2, whereby the point values resulting from the respective criteria are added.

### III. EXAMINATION OF THE MOTOR FUNCTION

18. Speech:

∇ 0—Normal.

∇ 1—Slight reduction in the expression, diction and/or volume.

∇ 2—Monotonous, faded, but understandable; moderately impaired.

∇ 3—Distinct impairment, difficult to understand.

∇ 4—Ununderstandable.

19. Facial Expression:

∇ 0—Normal.

∇ 1—Minimal alteration in facial play, could be a normal “Poker face”.

∇ 2—Slight, but clearly abnormal reduction in the facial expression.

∇ 3—Moderately diminished facial play; lips occasionally open.

∇ 4—Mask-like or frozen face with severe or complete absence of expression; Lips apart by about 7 mm.

20. Resting tremors: (G=Face, RH=Right hand, LH=Left hand, RF=Right foot, LF=Left foot)

G RH LH RF LF

∇ ∇ ∇ ∇ ∇ 0—None.

∇ ∇ ∇ ∇ ∇ 1—Slight and seldom.

∇ ∇ ∇ ∇ ∇ 2—Small amplitude persistent; or moderate amplitude, but only intermittent.

∇ ∇ ∇ ∇ ∇ 3—Moderate amplitude, present most of the time.

∇ ∇ ∇ ∇ ∇ 4—Pronounced amplitude, present most of the time.

21. Action or postural tremor of the hands: (R=Right, L=Left)

R L

∇ ∇ 0—Absent.

∇ ∇ 1—Slight; present with movement.

∇ ∇ 2—Moderate amplitude, present with movement.

∇ ∇ 3—Moderate amplitude, present with retention of the posture and with movement.

∇ ∇ 4—Pronounced amplitude; disturbing when eating.

22. Rigidity: (Examined for passive movement of the big joints with patient in sitting position. Cogwheel phenomenon can be ignored). (N=Neck, ROE=Right Upper Limb, LOE=Left Upper Limb,

RUE=Right Lower Limb, LUE=Left Lower Limb)

N ROE LOE RUE LUE

∇ ∇ ∇ ∇ ∇ 0—Absent.

∇ ∇ ∇ ∇ ∇ 1—Slight or recognizable only on activation of mirror-image or other movements.

∇ ∇ ∇ ∇ ∇ 2—Slight to moderate.

∇ ∇ ∇ ∇ ∇ 3—Pronounced, but full range of movement achievable.

∇ ∇ ∇ ∇ ∇ 4—Severe; difficulty in carrying out all movements.

23. Finger tapping: (Patient touches the thumb with index finger in quick sequence and with maximum possible amplitude and separately for each hand). (R=Right, L=Left).

R L

∇ ∇ 0—Normal.

∇ ∇ 1—Slight retardation and/or reduction of the amplitude.

∇ ∇ 2—Moderately restricted. Distinct and premature fatigue. Movement may be interrupted occasionally.

∇ ∇ 3—Severely restricted. Delayed start of the movements or interruption of the continuous movement.

∇ ∇ 4—Can hardly carry out the exercise.

24. Hand movement: (Patient opens and closes the hands in quick sequence with greatest possible amplitude and separately with each hand).

(R=Right, L=Left)

R L

∇ ∇ 0—Normal.

∇ ∇ 1—Slight retardation and/or reduction of the amplitude.

∇ ∇ 2—Moderately restricted. Distinct and premature fatigue. Movement may be interrupted occasionally.

∇ ∇ 3—Severely restricted. Delayed start of the movements or interruption of the continuous movement.

∇ ∇ 4—Can hardly carry out the exercise.

25. Rapidly alternating movement of the hands: (Pronation/Supination movement of the hands, vertical or horizontal, with largest possible amplitude, both hands at the same time).

R L

∇ ∇ 0—Normal.

∇ ∇ 1—Slight retardation and/or reduction of the amplitude.

∇ ∇ 2—Moderately restricted. Distinct and premature fatigue. Movement may be interrupted occasionally.

∇ ∇ 3—Severely restricted. Delayed start of the movements or interruption of the continuous movement.

∇ ∇ 4—Can hardly carry out the exercise.

26. Agility of the legs: (The patient knocks in rapid sequence with the heels on the floor and thereby lifts the whole leg. The amplitude should measure at least 7.5 cm.

R L

∇ ∇ 0—Normal.

∇ ∇ 1—Slight retardation and/or reduction of the amplitude.

∇ ∇ 2—Moderately restricted. Distinct and premature fatigue. Movement may be interrupted occasionally.

∇ ∇ 3—Severely restricted. Delayed start of the movements or interruption of the continuous movement.

∇ ∇ 4—Can hardly carry out the exercise.

27. Getting up from the chair: (The patient tries, with arms folded before the chest, to rise from a flat back rest wooden or metal chair).

∇ 0—Normal.

∇ 1—Slow; can need more than one attempt.

∇ 2—Pushes up on the armrests.

∇ 3—Tends to fall back and must try possibly several times, however can stand up without help.

∇ 4—Cannot stand up without help.

28. Posture:

∇ 0—Normal upright.

∇ 1—Not quite upright, posture slightly bent forward; could be normal for an elderly person.

∇ 2—Moderately bent forward posture; clearly abnormal, can be slightly inclined to one side.

∇ 3—Severely bent forward posture with kyphosis; can be moderately inclined to one side.

∇ 4—Pronounced bending with extremely abnormal posture.

29. Gait:

∇ 0—Normal.

∇ 1—Walks slowly, can drag along a few short steps, but without festination or propulsion.

∇ 2—Walks with difficulty, but needs little or no help; possibly slight mincing along, short steps or propulsion.

∇ 3—Severe disorder in walking, needs help.

∇ 4—Can not walk at all, not even with help.

30. Postural stability: (Reaction to sudden shifting backwards on pulling the shoulders of the patient; who stands straight, with open eyes and with the feet slightly apart. The patient is prepared for that).

∇ 0—Normal.

∇ 1—Retropulsion, compensates however without help.

∇ 2—Postural reaction absent; would fall, if he were not caught by the examiner.

∇ 3—Very unstable; tends to loss the balance spontaneously.

∇ 4—Can not stand without support.

31. Bradykinesia and hypokinesia of the body: (Combination of slowness, delay, reduced co-movement of the arms, less movement amplitude, and general hypokinesia)

∇ 0—None.

∇ 1—Minimal retardation, movement looks intentional; could be normal for some persons. Possibly diminished amplitude.

∇ 2—Slight retardation and hypokinesia, which are clearly abnormal. Alternatively also diminished amplitude.

∇ 3—Moderate retardation and hypokinesia or diminished amplitude.

∇ 4—Pronounced retardation, hypokinesia, or diminished amplitude.

### Working Example 6

### In Vitro Transformation of a Prodrug into Active Substance

From the liver cell homogenates of human, monkey, dog, rat, or mouse, a microsome fraction, containing the essential metabolizing enzymes, is obtained through differential centrifugation; alternatively, also the cytoplasmatic fraction can be obtained. The subcellular fraction is suspended with a buffer in such a manner that a solution with a defined protein content is obtained. After addition of 1 μM of the prodrug to be tested, incubation follows at 37° C. for 60 min. Finally, Rotigotine is quantified with the help of HPLC/UV or also of HPLC/MS and is put in relation to used quantities. For detailed analyses, the concentration or time series are investigated.

1-14. (canceled)

15. A method for treatment or prophylaxis of dopaminergic cell loss in a subject suffering from or susceptible to a disease associated with increased dopaminergic cell loss, comprising administration of rotigotine, or a salt or prodrug thereof to the subject.

16. The method of claim 15, wherein the dopaminergic cell loss is associated with Morbus Parkinson.

17. The method of claim 15, wherein prophylactic treatment is administered to a subject selected from the group consisting of:
(a) an individual not exhibiting symptoms of Morbus Parkinson, but having a high risk for developing Morbus Parkinson; and
(b) an individual exhibiting early symptoms of Morbus Parkinson, in whom at least three of four cardinal symptoms of Morbus Parkinson are not yet present or only partially present, said symptoms being selected from the group consisting of rigor, resting tremors, bradykinesia and postural instability.

18. The method of claim 15, wherein the subject has one or more clinical symptoms selected from the group consisting of smell disorder, depression, sleep disorder, REM Sleep Behavior Disorders, constipation and short duration movement anomalies.

19. The method of claim 15, wherein the subject exhibits a mutation in a PARK-gene and/or alterations in the alpha-synuclein or neuromelanin pattern.

20. The method of claim 15, wherein the subject shows a loss of 60% of dopaminergic cells in the substantia nigra prior to administration of the rotigotine, or a salt or prodrug thereof.

21. The method of claim 15, wherein the subject has a UPDRS score of less than 9 prior to administration of the rotigotine, or salt or prodrug thereof.

22. The method of claim 15, wherein the subject has a Höhn-Yahr score of 0 or 1.

23. The method of claim 15, wherein the rotigotine, or salt or prodrug thereof, is administered by parenteral, transdermal or mucosal administration.

24. The method of claim 15, wherein the rotigotine, or salt or prodrug thereof, is administered at a dose of 0.05 to 50 mg per day.

25. The method of claim 15, wherein the prodrug is selected from the group of esters consisting of carbamates, acetals, ketals, phosphates, phosphonates, sulfates, sulfonates and silylethers.

26. The method of claim 15, wherein the prodrug is selected from the group of carbonates consisting of, acyloxyalkyl ethers, thiocarbonyl esters, oxythiocarbonyl esters, thiocarbamates and ethers.

27. The method of claim 15, wherein rotigotine or salt thereof is administered to the subject.

28. The method of claim 15, wherein a rotigotine prodrug is administered to the subject.

29. A kit for diagnosis and preventive therapy of Morbus Parkinson comprising:
(a) a diagnostic agent that can enable diagnosis of Morbus Parkinson or of the predisposition to develop the Morbus Parkinson disease in the early or asymptomatic stage and
(b) a pharmaceutical formulation comprising rotigotine or a salt or prodrug thereof.

30. A kit according to claim 29, wherein the diagnostic agent (a) is selected from:
(i) an agent or a diagnosis kit for detection of neuromelanin;
(ii) an agent or a diagnosis kit for detection semaphorin 3;
(iii) an agent or a diagnosis kit for detection alpha-synuclein and/or its aggregates; or
(iv) an agent or a diagnosis kit for genetic evidence of the mutations connected with the occurrence of Morbus Parkinson and/or with an allele associated with the frequent occurrence of Morbus Parkinson.
